(19) United States
(12) Patent Application Publication
(10) Pub. No.: US 2013/0090466 A1
(43) Pub. Date: Apr. 11, 2013

(54) CRYSTALLIZATION PROCESS OF CYCLIC ADENOSINE 3',5'-MONOPHOSPHATE

(71) Applicants: Hanjie Ying, Nanjing City (CN); Wenbin Qian, Nanjing City (CN); Yong Chen, Nanjing City (CN); Xiaochun Chen, Nanjing City (CN); Jianxin Bai, Nanjing City (CN); Jian Xiong, Nanjing City (CN); Xiaoqing Lin, Nanjing City (CN); Jingjing Xie, Nanjing City (CN); Jinglan Wu, Nanjing City (CN)

(72) Inventors: Hanjie Ying, Nanjing City (CN); Wenbin Qian, Nanjing City (CN); Yong Chen, Nanjing City (CN); Xiaochun Chen, Nanjing City (CN); Jianxin Bai, Nanjing City (CN); Jian Xiong, Nanjing City (CN); Xiaoqing Lin, Nanjing City (CN); Jingjing Xie, Nanjing City (CN); Jinglan Wu, Nanjing City (CN)

(21) Appl. No.: 13/702,039

(22) PCT Filed: Apr. 21, 2011
(86) PCT No.: PCT/CN11/73121

§ 371 Date: Dec. 4, 2012

(30) Foreign Application Priority Data
Jun. 4, 2010 (CN) 201010191528.3

(51) Int. Cl. C07H 19/16

(52) U.S. Cl. CPC C07H 19/16
USPC 536/25.3

(57) ABSTRACT

Provided is a crystallization process of cyclic adenosine 3′,5′-monophosphate, which comprises the following steps: 1) reacting an aqueous solution of cyclic adenosine 3′,5′-monophosphate with a base to obtain a salt of cyclic adenosine 3′,5′-monophosphate; 2) reacting the cyclic adenosine 3′,5′-monophosphate salt solution obtained in step 1) with an acid to obtain cyclic adenosine 3′,5′-monophosphate; 3) keeping cyclic adenosine 3′,5′-monophosphate obtained in step 2) at 0-15° C.

### TECHNICAL FIELD

The present invention belongs to the field of crystallization technology and specifically relates to a crystallization method of 3′,5′-cyclic adenosine monophosphate.

### BACKGROUND TECHNOLOGIES

3′,5′-cyclic adenosine monophosphate, activator of protein kinase, derivative of nucleotide, is a kind of vital substance with physiological activity that wildly exists in human body. It is synthesized by adenosine triphosphate under catalysis of adenylate cyclase and regulates multiple functional activities in cells. As second messenger of hormones, it enables hormones to play roles in regulating physiological functions and substance metabolism in cells, change functions of cell membrane, facilitate calcium ion in sarcoplasmic reticulum of muscle to enter muscle fiber and thereby enhance myocardial contraction; it may also promote activities of oxidases of respiratory chain, ameliorate myocardial hypoxia, relieve syndromes of coronary heart disease and improve electrocardiogram. In addition, it plays an important role in regulating carbohydrate and fat metabolism, synthesis of nucleic acids, proteins and the like.

At present, there are few reports about studies on crystallization of cyclic adenosine monophosphate. Latest Preparation Techniques of Biochemical Drugs published in 2000 reports crystallization process of chemically-synthesized 3′,5′-cyclic adenosine monophosphate, comprising adding the same volume of 95% ethanol and adjusting pH to 1-2 with 2 mol/L hydrochloric acid to precipitate white crystal; filtering, gathering and drying the white crystal to obtain finished product cAMP;

recovering mother liquor; yield rate 62.48%. The fact that highly toxic reagents such as pyridine, phosphorus oxychloride and the like are added in the process of chemical synthesis and more by-products is produced due to incomplete reaction result in low yield rate of the subsequent crystal separation. Additionally, the Chinese Patent No. CN1702076A discloses crystallization from 3′,5′-cyclic adenosine monophosphate concentrate using freeze drying directly. However, freezing drying can only remove water in the solution and the yield rate is 100%, but cannot further improve purity of the product. Moreover, great investment in vacuum freeze-drying equipment and high cost of energy consumption limit industrial application of this technique.

### SUMMARY OF HE INVENTION

The dissociation equilibrium constant, pK, of the phosphate group of 3′,5′-cyclic adenosine monophosphate is 3.3-3.9. Existing form of the phosphate group will be changed by adjusting pH value of the solution. As shown in the following formulas, when pH>pK, the phosphate group of 3′,5′-cyclic adenosine monophosphate reacts with a base to produce 3′,5′-cyclic adenosine monophosphate salt; when pH<pK, 3′,5′-cyclic adenosine monophosphate salt reacts with an acid to produce 3′,5′-cyclic adenosine monophosphate.

cAMP·H⁺+NaOH=cAMP·Na⁺+H₂O

cAMP·Na⁺+HCl=cAMP·H⁺+NaCl

Thus, the objective of the present invention is to provide a new crystallization method with high yield for 3′,5′-cyclic adenosine monophosphate, utilizing crystallization techniques of combining reaction-and-low-temperature to replace enforced precipitation with solvent and freezing drying so as to overcome deficiencies of the current crystallization techniques for 3′,5′-cyclic adenosine monophosphate, such as low quality of product and low yield,

The objective of the present invention can be achieved by the technical schemes hereinafter. The present invention provides a crystallization method of 3′,5′-cyclic adenosine monophosphate. Said method comprises the following steps: 1) reacting 3′,5′-cyclic adenosine monophosphate aqueous solution with a base to produce 3′,5′-cyclic adenosine monophosphate salt solution; 2) reacting the 3′,5′-cyclic adenosine monophosphate salt solution produced in step 1) with an acid to produce 3′,5′-cyclic adenosine monophosphate; 3) preserving the 3′,5′-cyclic adenosine monophosphate produced in step 2) at 0-15° C., preferably at 0-10° C., and more preferably at 5-10° C. to obtain the expected product.

Preferably, in said step 1), 3′,5′-cyclic adenosine monophosphate aqueous solution is reacted with a base until the pH value of the solution reaches pH 6.0-10.0, preferably pH 6.0-9.0, more preferably pH 6.5-8.0.

Preferably, said base is one or more selected from ammonia water and sodium hydroxide, the concentration of which is 2-10 M, preferably 3-6 M, more preferably 3-5 M.

Preferably, the concentration of said 3′,5′-cyclic adenosine monophosphate aqueous solution in said step 1) is 15-350 g/L, preferably 150-200 g/L.

Preferably, the reaction temperature in said step 2) is 10-40° C., preferably 15-35° C., more preferably 30-35° C.

Preferably, in said step 2), 3′,5′-cyclic adenosine monophosphate salt aqueous solution is reacted with an acid until the pH value of the solution reaches pH 1.0-3.5, preferably pH 1.5-3.0, more preferably pH 2.0-2.5.

Preferably, said acid is one or more selected from sulfuric acid, hydrochloric acid and phosphoric acid, the concentration of which is 0.01-10 M, preferably 0.05-8 M, more preferably 2-4 M.

Preferably, in said step 2), acid is added slowly into the 3′,5′-cyclic adenosine monophosphate salt solution at a flow rate of 0.01-0.5%/min, preferably 0.04-0.4%/min, more preferably 0.1-0.4%/min of the volume of 3′,5′-cyclic adenosine monophosphate salt solution.

Preferably, in said step 2), the process of adding acid may also comprise step of stirring at a rate of 20-250 rpm, preferably 90-200 rpm, more preferably 100-150 rpm.

Preferably, the preserving time in said step 3) is 5-25 hours, preferably 5-20 hours, more preferably 18-20 hours.

Preferably, said method may also comprise suction filtration, lavation with ethanol and vacuum drying of the product obtained in step 3).

Preferably, said method also comprises the following steps: 1) reacting 3-5 M ammonia water or sodium hydroxide with 3′,5′-cyclic adenosine monophosphate aqueous solution in the concentration of 150-200 g/L to produce 3′,5′-cyclic adenosine monophosphate ammonium salt or sodium salt solution, where the pH value of the solution is pH 6.5-8.0; 2) reacting the 3′,5′-cyclic adenosine monophosphate salt solution produced in step 1) with an acid in the concentration of 2-4 M at 30-35° C. until the pH value of the solution reaches pH 2.0-2.5; 3) preserving the product obtained in step 2) at 5-10° C. for 18-20 hours, and after suction filtration, lavation with ethanol and vacuum drying to obtain the expected product. Preferably, the acid in said step 2) is added into the 3′,5′-cyclic adenosine monophosphate salt solution slowly at the flow rate of 0.1-0.4 ml/min; preferably, the process of adding acid may also comprise step of stirring at a rate of 100-150 rpm.

Furthermore, the present invention also provides a preparation method of 3′,5′-cyclic adenosine monophosphate, comprising the step of crystallizing 3′,5′-cyclic adenosine monophosphate aqueous solution by adopting the above-mentioned method.

The present invention may also be achieved by the following technical schemes. A crystallization method of 3′,5′-cyclic adenosine monophosphate comprises reacting 2-10 M ammonia water or sodium hydroxide with 3′,5′-cyclic adenosine monophosphate aqueous solution in the concentration of 15-350 g/L to produce 3′,5′-cyclic adenosine monophosphate ammonium salt or sodium salt solution with the pH value between 6 and 10, transferring the solution obtained to crystallization jar, maintaining the temperature at 10-40° C., controlling the stirring rate at 20-250 rpm, adding an acid in the concentration of 0.01 M-10.0 M slowly into the 3′,5′-cyclic adenosine monophosphate salt solution at a flow rate of 0.01-0.5%/min of the volume of 3′,5′-cyclic adenosine monophosphate aqueous solution to allow reaction for crystallization until the pH value of the aqueous solution reaches 1.0-3.5, stopping stirring, preserving the solution at 0-15° C. for 5-25 hours followed by suction filtration, lavation with ethanol and vacuum drying to obtain 3′,5′-cyclic adenosine monophosphate crystal with a purity higher than 99%. Wherein, the preferred concentration of ammonia water or sodium hydroxide is 3-6 M. Wherein, the pH value of 3′,5′-cyclic adenosine monophosphate ammonium salt or sodium salt solution is maintained between pH 6 and pH 9. Wherein, the preferred temperature is maintained at 15-35° C., the stirring rate is maintained at 90-200 rpm. Wherein, it is preferred that an acid in the concentration of 0.05 M-8.0 M is added at a flow rate of 0.1-0.4%/min of the volume of 3′,5′-cyclic adenosine monophosphate aqueous solution. The acid above is sulfuric acid, hydrochloric acid or phosphoric acid solution. Wherein, it is preferred that the reaction proceeds until the pH value of 3′,5′-cyclic adenosine monophosphate aqueous solution reaches 1.5-3.0 and the stirring is stopped. Wherein, it is preferred to preserve the solution at 0-10° C. for 5-20 hours.

The most preferred technical scheme is: reacting 3-5 M ammonia water or sodium hydroxide with 3′,5′-cyclic adenosine monophosphate aqueous solution in the concentration of 15-200 g/L to produce 3′,5′-cyclic adenosine monophosphate ammonium salt or sodium salt solution with the pH value between 6.5 and 8.0, transferring the solution obtained to crystallization jar, maintaining the temperature at 30-35° C., controlling the stirring rate at 100-150 rpm, adding an acid in the concentration of 2-4 M at a flow rate of 0.1-0.4%/min of the initial volume of 3′,5′-cyclic adenosine monophosphate aqueous solution until the pH value of the aqueous solution reaches 2.0-2.5, stopping stirring, preserving the solution at 5-10° C. for 18-20 hours followed by suction filtration, lavation with ethanol and vacuum drying to obtain 3′,5′-cyclic adenosine monophosphate crystal.

Therefore, comparing with conventional crystallization methods, the crystallization method provided in the present invention improves yield rate and quality of crystallization greatly, wherein the yield rate of crystallization product stabilizes at a level higher than 92% and the purity of the product is higher than 99%. Moreover, the crystallization system and preparation method provided in the present invention result in shorter operation time, inhibition of biodegradation of 3′,5′-cyclic adenosine monophosphate and accumulation of pigment impurity during crystallization process, no need for special devices for heating and cooling so as to save investment cost, easier control of operation process and better repeat repeatability. In summary, the crystallization method provided in the present invention improves quality of final product greatly and has high yield rate of crystallization, easy operation and good repeatability and thereby can apply to industrial production of 3′,5′-cyclic adenosine monophosphate.

The crystallization method provided in the present invention is particularly applicable to 3′,5′-cyclic adenosine monophosphate synthesized by fermentation method, that is, utilizing nucleotide metabolic pathway of microbes to synthesize 3′,5′-cyclic adenosine monophosphate from the substrate hypoxanthine after undergoing a series of reactions catalyzed by bio-enzymes, subjecting the fermentation broth of the synthesized 3′,5′-cyclic adenosine monophosphate firstly to pre-treatment and ion-exchange column chromatography and then to concentration by nanofiltration membrane to remove salts, transferring the concentrate into crystallization jar to allow crystallization. The synthesis method by microbes features on low cost, less pollution, fewer impurities, easier processes for isolation and purification and etc, combining the crystallization method provided in the present invention, makes such technique more applicable to industrial application.

### EMBODIMENTS OF THE INVENTION

The present invention may be better understood based on the examples hereinafter. However, it is understood easily by those skilled in the art that the particular ratios of materials, the technical conditions, and the results as depicted in the examples are only for illustrating the present invention, but do not intend to or shall not limit the present invention as described in details in the claims.

in each example and comparing example hereinafter, raw material 3′,5′-cyclic adenosine monophosphate can be prepared according to the method described below, but shall not be understood as a limitation of the present invention. Any 3′,5′-cyclic adenosine monophosphate obtained commercially may be crystallized by using the method provided in the present invention.

1) Fermentation: Arthrobacterium A302 (deposited in China General Microbiological Culture Collection Center (CGMCC for short) on 18 January 2010; Address of the depository authority: Institute of Microbiology, Chinese Academy of Sciences, Datun Road, Chaoyang District, Beijing; Deposit No.: CGMCC No. 3584) is inoculated in a seed medium (calculated based on weight percentage of the medium, comprising 1% glucose, 1% peptone, 0.5% yeast extract, 1% beef extract, and 0.3% NaCl) with the initial pH of 7.0, incubating the inoculum at 30° C. and 240 rpm for 18 hours; the culture is inoculated in an inoculation size of 10% into fermentation medium (calculated based on weight percentage of the medium, comprising 5% glucose, 1% K₂HPO₄, 1% KH₂PO₄, 1% MgSO₄, 0.5% urea and 0.5% peptone, plus addition of 0.1 g NaF, 0.1 g VB1 and 5 g hypoxanthine per liter of the medium) in a 5 L fermentation tank; the pH value is controlled at 7.0 using NaOH and oxygen dissolution is controlled at 30%; the inoculated medium is fermented at 30° C. and 400 rpm for 72 hours. The yield of adenosine cyclophosphate is 5.0-10.0 g/L, white discharging the tank. The fermentation broth is centrifuged in a centrifuge to remove thallus. To remove majority of proteins, the supernatant is then passed through an ultrafiltration membrane that intercepts proteins whose molecular weight is larger than 6,000 Dalton to obtain a 3′,5′-cyclic adenosine monophosphate clear liquid in the concentration of 5.0-10.0 g/L.

2) Isolation and purification: a fixed bed is charged with 500 g of anion exchange resin (Amberlite IRA900RF Class Cl); after equilibrium, the 3′,5′-cyclic adenosine monophosphate clear liquid in the concentration of 5.31 g/L is loaded onto the column and the adsorption saturation is achieved after loading 8.098 L of the clear liquid; the column is washed with 0.25 mol/L ammonia water followed by eluted with 0.3 mol/L HCl for 16 hours. The volume of the eluent obtained is 14.048 L and the concentration is 2.96 g/L. The purity of the 3′ -cyclic adenosine monophosphate obtained is 95.0% and the yield rate is 96.7%.

3) Coarse crystallization: 3-5 M ammonia water or sodium hydroxide is reacted with 3′,5′-cyclic; adenosine monophosphate aqueous solution in the concentration of 150-200 g/L to produce 3′,5′-cyclic adenosine monophosphate ammonium salt or sodium salt solution with the pH value between 6.5 and 8.0; the solution obtained is transferred to a crystallization jar while maintaining the temperature at 30-35° C. and controlling the stirring rate at 100-150 rpm, adding an acid in the concentration of 2-4 M at a flow rate of 0. 1-0.4%/mm of the initial volume of 3′,5′-cyclic adenosine monophosphate aqueous solution until the p1-1 value of the aqueous solution reaches 2.0-2.5, followed by stopping stirring; the solution is preserved at 5-10° C. for 18-20 hours followed by suction filtration, lavation with ethanol and vacuum drying to obtain 3′,5′-cyclic adenosine monophosphate crystal with a purity of 97-98%.

Best conditions of high performance liquid chromatography for detecting 3′,5′-cyclic adenosine monophosphate are:

Chromatographic column: Hanbang Lichrospher-5-C18 chromatographic column (250 mm×4.6 mm i.d., 5 μm); mobile phase: methanol-6% (volume fraction) phosphoric acid aqueous solution (adjusting the pH value to 6.6 with triethylamine) (volume ratio of 25:75); flow rate: 0.8 mL/min; wave length for detection: 255 nm; column temperature: room temperature; injection volume: 20 μL. One point external standard method is used for quantification.

In the examples hereinafter, crystallization yield is calculated by firstly dividing the weight of the 3′,5′-cyclic adenosine rnonophosphate obtained by final crystallization by the weight of the 3′,5′-cyclic adenosine monophosphate charged into the crystallization jar, i.e. the fed 3′,5′-cyclic adenosine monophosphate, and then multiplying by 100%; while purity is calculated by firstly dividing peak area of 3′,5′-cyclic adenosine monophosphate detected by high performance liquid chromatography by total area of all the peaks and then multiplying by 100%.

### EXAMPLE 1

5 M ammonia water was reacted with 3′,5′-cyclic adenosine monophosphate aqueous solution to produce 0.95 L of 3′,5′-cyclic adenosine monophosphate ammonium salt solution of the concentration of 187 g/L while the pH was controlled at pH 8.0. The solution obtained was charged into a crystallization jar. The stirring rate was controlled at 150 rpm at 30° C. Phosphoric acid in the concentration of 2 M was added slowly at a flow rate of 0.2%/min of the initial volume of 3′,5′-cyclic adenosine monophosphate ammonium salt solution (i.e. 1.9 mL/min). The addition of the acid was stopped until the pH value reached 2.0. The solution was cooled to 5° C. and then preserved for 20 hours. After the crystallization was completed, the suspension liquid was filtered by suction and the white crystal obtained was washed with ethanol and then dried under vacuum to obtain 166.11 g of 3′,5′-cyclic adenosine monophosphate crystal with a crystallization yield of 93.5% and a purity of 99.4%.

### EXAMPLE 2

3 M ammonia water was reacted with 3′,5′-cyclic adenosine monophosphate aqueous solution to produce 1 L of 3′,5′-cyclic adenosine monophosphate ammonium salt solution of the concentration of 105 g/L while the pH was controlled at pH 7.0. The solution obtained was charged into a crystallization jar. The stirring rate was controlled at 200 rpm at 35° C. Sulfuric acid in the concentration of 3 M was added slowly at a flow rate of 0.4%/min of the initial volume of 3′,5′-cyclic adenosine monophosphate ammonium salt solution (i.e. 4 mL/min). The addition of the acid was stopped until the pH value reached 2.5. The solution was cooled to 5° C. and then preserved for 18 hours. After the crystallization was completed, the suspension liquid was filtered by suction and the white crystal obtained was washed with ethanol and then dried under vacuum to obtain 96.71 g of 3′,5′-cyclic adenosine monophosphate crystal with a crystallization yield of 92.10% and a purity of 99.4%.

### EXAMPLE 3

3 M sodium hydroxide was reacted with 3′,5′-cyclic adenosine monophosphate aqueous solution to produce 1.5 L of 3′,5′-cyclic adenosine monophosphate sodium salt solution of the concentration of 185 g/L while the pH was controlled at pH 8.0. The solution obtained was charged into a crystallization jar. The stirring rate was controlled at 200 rpm at 30° C. Sulfuric acid in the concentration of 2 M was added slowly at a flow rate of 0.4%/min of the initial volume of 3′,5′-cyclic adenosine monophosphate sodium salt solution (i.e. 6 mL/min), The addition of the acid was stopped until the pH value reached 2.0. The solution was cooled to 5° C., and then preserved for 20 hours. After the crystallization was completed, the suspension liquid was filtered by suction and the white crystal obtained was washed with ethanol and then dried under vacuum to obtain 256.97 g of 3′,5′-cyclic adenosine monophosphate crystal with a crystallization yield of 92.6% and a purity of 99.3%.

### COMPARING EXAMPLE 1

5 M ammonia water was reacted with 3′,5′-cyclic adenosine monophosphate aqueous solution to produce 0.95 L of 3′,5′-cyclic adenosine monophosphate ammonium salt solution of the concentration of 187 g/L while the pH was controlled at pH 8.0. The solution Obtained was charged into a crystallization jar. The stirring rate was controlled at 150 rpm at 30° C. Phosphoric acid in the concentration of 2 M was added slowly at a flow rate of 0.2%/min of the initial volume of 3′,5′-cyclic adenosine monophosphate ammonium salt solution (i.e. 1.9 mL/min). The addition of the acid was stopped until the pH value reached 2.0. The solution was preserved at 25° C. for 20 hours. After the crystallization was completed, the suspension liquid was filtered by suction and the white crystal obtained was washed with ethanol and then dried under vacuum to obtain 153.8 g of 3′,5′-cyclic adenosine monophosphate crystal with a crystallization yield of 86.6% and a purity of 99.2%.

### COMPARING EXAMPLE 2

3 M sodium hydroxide was reacted with 3′,5′-cyclic adenosine monophosphate aqueous solution to produce 2 L of 3′,5′-cyclic adenosine monophosphate sodium salt solution of the concentration of 187 g/L while the pH was controlled at pH 8.0. The solution obtained was charged into a crystallization jar, The stirring rate was controlled at 100 rpm at 40° C. Hydrochloric acid in the concentration of 3 M was added slowly at a flow rate of 0.1%/min of the initial volume of 3′,5′-cyclic adenosine monophosphate sodium salt solution (i.e. 2 mL/min). The addition of the acid was stopped until the pH value reached 3.0. The solution was cooled to 5° C., and then preserved for 20 hours. After the crystallization was completed, the suspension liquid was filtered by suction and the white crystal obtained was washed with ethanol and then dried under vacuum to obtain 319.32 g of 3′,5′-cyclic adenosine monophosphate crystal with a crystallization yield of 85.38% and a purity of 98.3%.

1-10. (canceled)

11. A crystallization method of 3′,5′-cyclic adenosine monophosphate, wherein said method comprises the following steps:
1) reacting 3′,5′-cyclic adenosine monophosphate aqueous solution with a base to produce 3′,5′-cyclic adenosine monophosphate salt solution;
2) reacting the 3′,5′-cyclic adenosine monophosphate salt solution produced in step 1) with an acid to produce 3′,5′-cyclic adenosine monophosphate; and
3) preserving the 3′,5′-cyclic adenosine monophosphate produced in step 2) at 0-15° C.

12. The method according to claim 11, wherein in said step 1), 3′,5′-cyclic adenosine monophosphate aqueous solution is reacted with a base until the pH value of the solution reaches pH 6.0-10.0.

13. The method according to claim 12, wherein said base is one or more selected from ammonia water and sodium hydroxide, the concentration of which is 2-10 M.

14. The method according to claim 12, wherein the concentration of said 3′,5′-cyclic adenosine monophosphate aqueous solution is 15-350 g/L.

15. The method according to claim 11, wherein in said step 2), 3′,5′-cyclic adenosine monophosphate salt solution is reacted with an acid until the pH value of the solution reaches pH 1.0-5.0.

16. The method according to claim 15, wherein said acid is one or more selected from sulfuric acid, hydrochloric acid and phosphoric acid, the concentration of which is 0.01-10 M.

17. The method according to claim 11, wherein the reaction temperature in said step 2) is 10-40° C.

18. The method according to claim 11, wherein in said step 2), the acid is added slowly into the 3′,5′-cyclic adenosine monophosphate salt solution at a flow rate of 0.01-0.5%/min of the volume of 3′,5′-cyclic adenosine monophosphate salt solution.

19. The method according to claim 18, wherein the process of adding acid comprises step of stirring at a stirring rate of 20-250 rpm.

20. The method according to claim 11, wherein the preserving time in said step 3) is 5-25 hours.

21. The method according to claim 11, wherein said method also comprises suction filtration, lavation with ethanol and vacuum drying of the product obtained in step 3).

22. The method according to claim 11, wherein said method also comprises the following steps:
1) reacting 3-5 M ammonia water or sodium hydroxide with 3′,5′-cyclic adenosine monophosphate aqueous solution in the concentration of 150-200 g/L to produce 3′,5′-cyclic adenosine monophosphate ammonium salt or sodium salt solution, where the pH value of the solution is pH 7.5-8.5;
2) reacting the 3′,5′-cyclic adenosine monophosphate salt solution produced in step 1) with an acid in the concentration of 2-4 M at 30-35° C. until the pH value of the solution reaches pH 2.0-2.5; and
3) preserving the product obtained in step 2) at 5-10° C. for 18-20 hours, and after suction filtration, lavation with ethanol and vacuum drying to obtain the expected product.

23. The method according to claim 22, wherein the acid in said step 2) is added into the 3′,5′-cyclic adenosine monophosphate salt solution slowly at a flow rate of 0.1-0.4%/min of the volume of 3′,5′-cyclic adenosine monophosphate salt solution.

24. The method according to claim 23, wherein the process of adding acid also comprises step of stirring at a rate of 100-150 rpm.

25. A preparation method of 3′,5′-cyclic adenosine monophosphate, wherein said method comprising a step of crystallizing 3′,5′-cyclic adenosine monophosphate by utilizing said method according to claim 11.
